(19) United States
(12) Patent Application Publication
(10) Pub. No.: US 2017/0086901 A1
(43) Pub. Date: Mar. 30, 2017

(54) CATHETER FOR RENAL DENERVATION

(71) Applicant: CRYOMEDIX, LLC, San Diego, CA (US)

(72) Inventors: William J. Nydam, Las Vegas, NV (US); Barron W. Nydam, San Diego, CA (US)

(21) Appl. No.: 15/263,666

(22) Filed: Sep. 13, 2016

Related U.S. Application Data
(60) Provisional application No. 62/234,476, filed on Sep. 29, 2015.

(51) Int. Cl. A61B 18/02

(52) U.S. Cl. CPC A61B 18/02; A61B 2018/00041

(57) ABSTRACT

A catheter having a cryogenic section at its distal end is provided to perform a neuromodulation of a renal artery. When positioned in the artery, the cryogenic section can be reconfigured from a tube-like configuration into a helical configuration. When in its helical configuration, the cryogenic section makes contact with the inner wall of the renal artery, along a predetermined length, through a 360° pitch. Cryogenic fluid is then introduced into the cryogenic section to neuromodulate the renal artery.

This application claims the benefit of U.S. Provisional Patent Application Ser. No. 62/234,476, filed Sep. 29, 2015. The entire contents of Application Ser. No. 62/234,476 are hereby incorporated by reference herein.

### FIELD OF THE INVENTION

The present invention generally pertains to cryogenic catheters that are useful for neuromodulation of arterial tissue in the vasculature of a patient. More particularly, the present invention pertains to cryogenic catheters which can be reconfigured, in situ, inside the lumen of an artery to perform a circumferential neuromodulation at the wall of the lumen. The present invention is particularly, but not exclusively, useful as a cryogenic catheter for performing a neuromodulation of a renal artery.

### BACKGROUND OF THE INVENTION

Renal hypertension is caused by a kidney disease which, in a number of cases, is characterized by a narrowing of the renal artery. Because this narrowing of the renal artery results in lower blood flow into a kidney, the kidney responds with a hormonal reaction that creates a demand for more water and salt in the body. The resultant increase in hydration contributes to hypertension. Heretofore, a typical treatment for renal hypertension has involved the use of drugs. However, another cause of hypertension is due to hyperactivity of the nerves. The present invention, however, understands that a neuromodulation of the renal artery can minimize or eliminate renal hypertension.

Neuromodulation is a technology that acts directly upon nerves and their activity in the neurovascular system of a patient. As envisioned by the present invention, cryoablation techniques can be employed to disrupt unwanted nerve activity in the nerves surrounding the lumen of a renal artery.

Anatomically, the renal artery has a relatively large lumen. Thus, an effective cryoablation in the lumen of a renal artery requires a cryo-probe that can establish circumferential contact around the wall of the renal artery. A consequence of this requirement, however, is the problems that are encountered when advancing and maneuvering a probe of sufficient size, through the vasculature of a patient, and into position for circumferential contact with the wall of a renal artery.

In light of the above, it is an object of the present invention to provide a cryogenic catheter for performing a neuromodulation in a renal artery of a patient for the purpose of treating hypertension. Another object of the present invention is to provide a cryogenic catheter that has a minimal profile diameter during a maneuvering and positioning of the catheter in the vasculature of a patient, but which can be reconfigured into a helical configuration with a greater profile diameter once the catheter is properly positioned in the lumen of a renal artery. Still another object of the present invention is to provide a cryogenic catheter for neuromodulating a renal artery to treat hypertension which is easy to use, is relatively simple to manufacture, and is comparatively cost effective.

### SUMMARY OF THE INVENTION

In accordance with the present invention, a system and method for performing a circumferential neuromodulation essentially includes a catheter with a cryo-section that is affixed to the distal end of the catheter. In particular, the import of the present invention is the capability of the cryo-section to be reconfigured after it has been positioned inside the lumen of a renal artery of a patient for the treatment of hypertension.

For purposes of the present invention, prior to the cryo-section being positioned in the renal artery, the cryo-section is in a stressed state and it is configured as an elongated tube extending along a longitudinal axis. In this stressed state, a portion of the cryo-section extends through a length L_{tube} that is established between two predetermined points along the longitudinal axis. Once it has been properly positioned in the artery, however, the cryo-section is converted to an unstressed state wherein it is reconfigured as a helix that has expanded into contact with the inner wall (adventitia) surrounding the lumen of the renal artery. In the unstressed state (i.e. its helical configuration), the cryo-section defines a helix axis and the distance between the two predetermined points on the longitudinal axis define a length Lₕₑₗᵢₓ along the helix axis (comparatively, Lₕₑᵢₗₓ<L_{tube}). A cryogenic fluid is then introduced into the cryo-section to cryoablate tissue and nerves in the wall (adventitia) of the renal artery. The consequent freezing effect thus accomplishes the purpose of the present invention.

Structurally, the cryo-section of the present invention is generally tube-shaped and it is made of a suitable material which, as noted above, is formed in its unstressed state as a helix. The cryo-section is also formed with a fluid chamber and a stiffening lumen. Both the fluid chamber and the stiffening lumen extend longitudinally in the cryo-section through a distance that is greater than a predetermined length L_{tube}. Additionally, detectable markers can be placed on the external surface of the cryo-section to assist in its proper placement for a treatment protocol. As noted above, the cryo-section is affixed to the distal end of the catheter.

Also included with the present invention is a longitudinal stiffening wire that interacts with the stiffening lumen of the cryo-section. It is through this interaction that reconfigurations of the cryo-section are affected. In detail, the longitudinal stiffening wire can be selectively extended distally from the proximal end of the catheter, and thereby inserted into the stiffening lumen of the cryo-section. When so inserted, the longitudinal stiffening wire holds the cryo-section in its stressed state wherein it is configured as an elongated tube. Upon withdrawal of the longitudinal stiffening wire from the stiffening lumen of the cryo-section, however, the cryo-section returns to its unstressed state wherein it is configured as a helix.

As intended for the present invention, the helix configuration for the cryo-section will have a pitch of 360° along the predetermined length Lₕₑₗᵢₓ, noted above. Functionally, when the cryo-section is expanded into contact with the inner wall that surrounds the lumen of the renal artery, it is in its helix configuration. Importantly, this contact between the cryo-section and the inner wall of the renal artery will be continuous and uninterrupted through the 360° pitch along the length Lₕₑₗᵢₓ of the helix. It is also important that the fluid chamber inside the cryo-section also extend through the 360° pitch along the length Lₕₑₗᵢₓ of the helix. Consequently, all nerves in the wall (adventitia) of the renal artery along the length Lₕₑₗᵢₓ of the renal artery will be subject to cryoablation and neuromodulation.

Operationally, cryoablation and neuromodulation for the present invention are accomplished when cryogenic fluid is introduced into the fluid chamber of the cryo-section. This, of course, is done while the cryo-section is configured as a helix in the renal artery. After the cryoablation and neuromodulation of the renal artery have been completed, the system of the present invention is removed from the vasculature of the patient. This removal is accomplished by first warming the cryo-section to separate it from the frozen tissue. The longitudinal stiffening wire can then be used to re-stiffen the cryo-section. Thus, the cryo-section is again stressed and is configured as an elongated tube to facilitate removal of the system from the vasculature of a patient.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a system in accordance with the present invention as it is being employed in an intended operational environment;

FIG. 2 is an anatomical representation of a renal artery with the cryo-section of the present invention operationally positioned in the lumen of the renal artery;

FIG. 3 is a cross-section view of the renal artery as seen along the line 3-3 in FIG. 2;

FIG. 4A shows the cryo-section of the present invention in a stressed state wherein it is configured as a substantially straight, elongated tube;

FIG. 4B shows the cryo-section as seen in FIG. 4A in an unstressed state wherein it is configured as a helix;

FIG. 5 is a cross-section view of the cryo-section as seen along the line 5-5 in FIG. 4A; and

FIG. 6 is a cross-section view of the cryo-section as seen along the line 6-6 in FIG. 4A.

### DESCRIPTION OF THE PREFERRED EMBODIMENTS

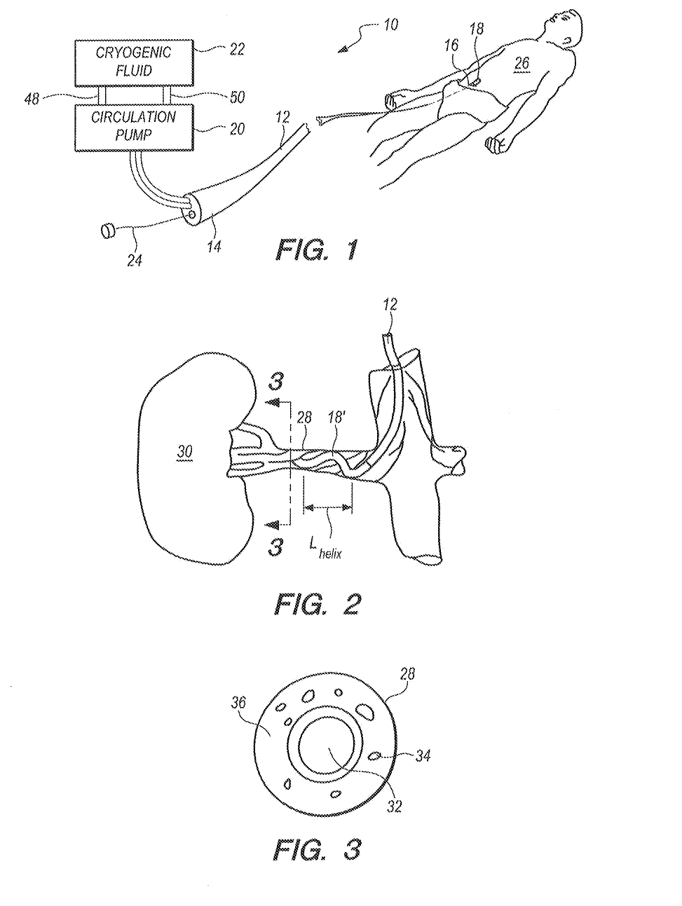

Referring initially to FIG. 1, a system for performing a circumferential neuromodulation in accordance with the present invention is shown and is generally designated 10. As shown, the system 10 includes a catheter 12 that has a proximal end 14 and a distal end 16. Additionally, the system 10 includes a cryo-section 18 that is affixed to the distal end 16 of the catheter 12. The system 10 also includes a circulation pump 20 that is connected in fluid communication with the catheter 12 at its proximal end 14 for the purpose of pumping fluid from a cryogenic fluid source 22 into the cryo-section 18 of the catheter 12. As disclosed below, in detail, the primary purpose of the present invention is to perform a circumferential neuromodulation in the vasculature of a patient 26.

FIG. 2 indicates that for an operation of the system 10, the catheter 12 will be advanced through the vasculature of a patient 26 to position the cryo-section 18 of the catheter 12 in a renal artery 28 of a kidney 30. A circumferential neuromodulation in the lumen 32 of the renal artery 28 can then be performed in accordance with the present invention. In particular, this neuromodulation is performed by cryoablating nerves 34 in the adventitia 36 of the renal artery 28 (see FIG. 3). As envisioned for the present invention, advancement of the catheter 12 with the cryo-section 18 into the renal artery 28 through the vasculature of the patient 26 can be accomplished in any manner well known in the pertinent art. For instance, a mechanism such as a guiding catheter (not shown) can be used for this purpose.

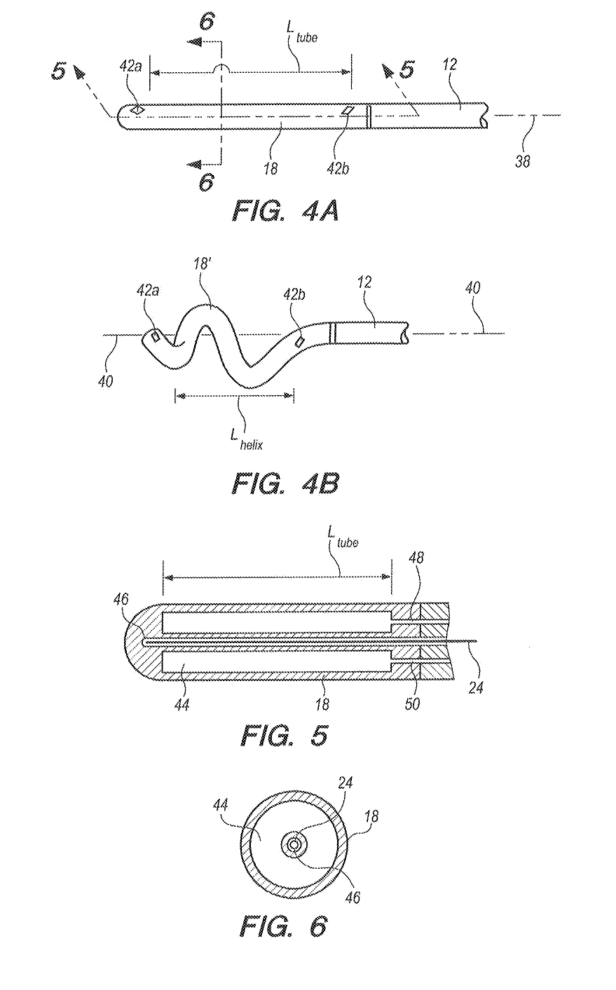

The functional capabilities of the cryo-section 18 of catheter 12 are best appreciated with reference to both FIGS. 4A and 4B. In FIG. 4A, the cryo-section 18 is shown in its stressed state, wherein it is configured as an elongated tube that extends along a longitudinal axis 38. On the other hand, FIG. 4B shows the cryo-section 18′ in its unstressed state wherein it is configured as a helix that extends around a helix axis 40. As a practical matter, the longitudinal axis 38 and the helix axis 40 are essentially collinear. Importantly, the pitch of the cryo-section 18′ will be at least 360° through the length Lₕₑₗᵢₓ on the helix axis 40. As noted above, Lₕₑₗᵢₓ will necessarily be less than L_{tube}. Further, both FIGS. 4A and 4B show that a marker 42a and a marker 42b are positioned on the cryo-section 18, and FIG. 4A indicates that the markers 42a and 42b will straddle the length L_{tube} when the cryo-section 18 is in its stressed configuration.

In FIG. 5 it will be seen that the cryo-section 18 is formed with a fluid chamber 44. Also, with cross reference to FIG. 6, it is seen that a stiffening lumen 46 is centered in the fluid chamber 44 and is dimensioned to receive the stiffening wire 24. Further, it is to be appreciated that a supply line 48 is created in the catheter 12 to establish fluid communication between the cryogenic fluid source 22 and the fluid chamber 44. Similarly, a return line 50 is created for the catheter 12 which, like the supply line 48 establishes fluid communication between the cryogenic fluid source 22 and the fluid chamber 44.

For an operation of the system 10, the methodology for performing a circumferential neuromodulation in accordance with the present invention first requires inserting the stiffening wire 24 into the stiffening lumen 46 of the cryo-section 18 to configure the cryo-section 18 in its stressed configuration (see FIG. 4A). The cryo-section 18 of catheter 12 is then advanced through the vasculature of patient 26 to position the cryo-section 18 in the lumen 32 of a renal artery 28 of the patient 26. Importantly, this advancement of the cryo-section 18 is accomplished while the cryo-section 18 is in a stressed configuration having a shape of an elongated tube and it is facilitated by monitoring the markers 42a and 42b.

Once the cryo-section 18 is properly positioned in the renal artery 28 the stiffening wire 24 is withdrawn from the stiffening lumen 46. With this withdrawal of the stiffening wire 24, the cryo-section 18 is reconfigured into its unstressed configuration (see FIG. 4B). As disclosed above, in its unstressed configuration the cryo-section 18 is formed as a helix which is centered on the helix axis 40, and it will have a pitch of 360° along the predetermined length Lₕₑₗᵢₓ.

When the helically configured cryo-section 18′ has been established in the renal artery 28, a cryogenic fluid from the cryogenic fluid source 22 is introduced into the fluid chamber 44 of the cryo-section 18′ through the supply line 48. The cryogenic fluid is then removed from the fluid chamber 44 through the return line 50. As envisioned for the present invention, within this cooperative combination of structure, the cryogenic fluid can be continuously recycled through the system 10 until the circumferential neuromodulation of the renal artery 28 is completed. When the operation has been completed the cryo-section 18′ can be warmed to release it from frozen tissue in the renal artery 28, to thereby facilitate the removal of the cryo-section 18 from the vasculature of the patient 26.

Once the circumferential neuromodulation has been completed, and the cryo-section 18 has been warmed, the stiffening wire 24 can be reinserted into the stiffening lumen 46 to thereby reconfigure the cryo-section 18 as an elongated tube. Finally, the cryo-section 18 and the catheter 12 are withdrawn from the vasculature of the patient.

While the particular Catheter for Renal Denervation as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for performing a circumferential neuromodulation which comprises:
a source of a cryogenic fluid;
a catheter having a proximal end and a distal end;
a tubular-shaped cryo-section affixed to the distal end of the catheter;
a fluid chamber formed in the cryo-section for receiving cryogenic fluid from the source of cryogenic fluid;
a means for transitioning the cryo-section between a stressed state wherein the cryo-section is configured as an elongated tube characterized by a longitudinal axis, and an unstressed state wherein the cryo-section is configured as a helix characterized by a helix axis;
a steering mechanism mounted on the catheter, for guiding the cryo-section into a lumen in an artery of the vasculature of a patient; and
a circulation pump for introducing the cryogenic fluid from the source of cryogenic fluid and into the fluid chamber of the cryo-section to perform a circumferential neuromodulation when the cryo-section is configured as a helix in its unstressed state.

2. The system recited in claim 1 wherein the cryo-section, when configured as the elongated tube in its stressed state, extends the fluid chamber through a length L_{tube} along the longitudinal axis of the cryo-section.

3. The system recited in claim 2 wherein the cryo-section, when configured as the helix in its unstressed state, has a pitch greater than 360° through a predetermined length Lₕₑₗᵢₓ along the helix axis of the cryo-section, and wherein Lₕₑₗᵢₓ is less than L_{tube}.

4. The system recited in claim 3 wherein Lₕₑₗᵢₓ is predetermined to establish a circumferential contact between the cryo-section and a wall of the lumen of the artery.

5. The system recited in claim 1 wherein a stiffening lumen is formed in the cryo-section, and wherein the means for transitioning the cryo-section comprises a longitudinal stiffening wire for insertion into the stiffening lumen.

6. The system recited in claim 5 wherein the cryo-section is in its stressed state and its elongated tube configuration when the longitudinal stiffening wire is inserted into the stiffening lumen of the cryo-section, and the cryo-section is in its unstressed state and its helix configuration when the longitudinal stiffening wire is withdrawn from the stiffening lumen of the cryo-section.

7. The system recited in claim 6 wherein the cryo-section is in its stressed state and its elongated tube configuration during an advancement and during a withdrawal of the cryo-section from the vasculature of the patient.

8. The system recited in claim 1 wherein the cryo-section has an outer surface and at least one detectable marker is positioned on the outer surface to assist in guiding and positioning the cryo-section in the vasculature of the patient.

9. The system recited in claim 1 wherein the catheter is formed with a fluid supply line connecting the fluid chamber of the cryo-section in fluid communication with the proximal end of the catheter, and with a fluid return line connecting the fluid chamber of the cryo-section in fluid communication with the proximal end of the catheter, and wherein the circulation pump is connected in fluid communication with the fluid supply line and the fluid return line for recirculating fluid through the system between the source of cryogenic fluid and the cryo-section of the catheter.

10. The system recited in claim 1 wherein the circulation pump includes a means for warming the cryo-section to release the cryo-section from frozen tissue in the artery for removal of the cryo-section from the vasculature of the patient.

11. A method for performing a circumferential neuromodulation which comprises the steps of:
advancing a cryo-section of a catheter through the vasculature of a patient to position the cryo-section in a lumen of a renal artery of the patient, wherein the cryo-section is formed with a fluid chamber, and wherein the advancing step is accomplished while the cryo-section is in a stressed configuration having a shape of an elongated tube with the fluid chamber extending through a length L_{tube} along the longitudinal axis of the cryo-section;
reconfiguring the cryo-section into an unstressed configuration once the cryo-section is positioned in the lumen of the renal artery, wherein the cryo-section in its unstressed configuration is formed as a helix centered on a helix axis defined by the cryo-section, with the helix having a pitch of 360° along a predetermined length Lₕₑₗᵢₓ of the helix axis, wherein the helix axis is collinear with the longitudinal axis and Lₕₑₗᵢₓ is less than L_{tube}; and
introducing a cryogenic fluid into the fluid chamber formed in the cryo-section, wherein the introducing step is accomplished after the reconfiguring step and is performed to cryoablate tissue of the renal artery in contact with the helix along the length Lₕₑₗᵢₓ relative to the fluid chamber to perform the circumferential neuromodulation.

12. The method recited in claim 11 wherein the cryo-section is formed with a stiffening lumen, and the method further comprises the step of inserting a stiffening wire into the stiffening lumen to configure the cryo-section in its stressed configuration prior to the advancing step.

13. The method recited in claim 12 wherein the reconfiguring step is accomplished by withdrawing the stiffening wire from the stiffening lumen.

14. The method recited in claim 13 further comprising the step of reinserting the stiffening wire into the stiffening lumen after the introducing step, and the method further comprises the step of removing the cryo-section from the vasculature of the patient.

15. The method recited in claim 11 further comprising the step of monitoring a detectable marker on the cryo-section during the advancing step.

16. The method recited in claim 11 wherein the catheter is formed with a fluid supply line connecting the fluid chamber of the cryo-section in fluid communication with the proximal end of the catheter, and with a fluid return line connecting the fluid chamber of the cryo-section in fluid communication with the proximal end of the catheter, and the method further comprises the step of recirculating the cryogenic fluid through the fluid chamber during the introducing step.

17. The method recited in claim 11 further comprising the step of warming the cryo-section to release the cryo-section from frozen tissue in the artery for removal of the cryo-section from the vasculature of the patient.
